(19) United States
(12) Patent Application Publication
(10) Pub. No.: US 2024/0206781 A1
(43) Pub. Date: Jun. 27, 2024

(54) OXYMETRY DEVICE AND METHOD FOR MEASURING FETUS OXYGEN LEVELS DURING LABOR

(71) Applicant: SIGNALIFE LTD, Tel-Aviv (IL)

(72) Inventors: Roy NISSIM, Tel-Aviv (IL); Roy MIZRACHI, Haifa (IL)

(73) Assignee: SIGNALIFE LTD, Tel-Aviv (IL)

(21) Appl. No.: 18/038,996

(22) PCT Filed: Nov. 28, 2021
(86) PCT No.: PCT/IL2021/051413

§ 371 Date: May 26, 2023

Related U.S. Application Data
(60) Provisional application No. 63/118,765, filed on Nov. 27, 2020.

(51) Int. Cl. A61B 5/1464; A61B 5/00; A61B 5/024

(52) U.S. Cl. CPC A61B 5/1464; A61B 5/02411; A61B 5/02433; A61B 5/7221; A61B 5/7235; A61B 5/743; A61B 5/6814; A61B 2503/02; A61B 2560/0462; A61B 2562/0204; A61B 2562/0219; A61B 2562/0233; A61B 2562/0247; A61B 2562/0271

(57) ABSTRACT

An oximetry device for determining mammalian fetus oxygen levels, the oximetry device constituted of: a sensor module comprising multiple light sources of different wavelengths and at least one optical sensor; a processing unit in communication with the sensor module, the processing unit comprising a processor, and a memory, the processor in communication with an output device, the memory comprising electronically readable instructions to cause the processor to: energize the multiple light sources; determine a time varying amplitude of an output of the at least one optical sensor; asses a quality of a location of the sensor module responsive to the determined time varying amplitude; provide to the output device an indication of the assessment of quality of the location; extract features from the determined time varying amplitude; and output an indication of the mammalian fetus oxygen level responsive to the extracted features from the determined time varying amplitude.

### TECHNICAL FIELD

The present invention relates generally to a device and a method for determining mammalian fetus oxygen levels during labor.

### BACKGROUND

During labor, it is important to monitor the health of a fetus to ensure a positive outcome. Electronic fetal heart rate monitoring is routinely used to assess fetal well being, or more precisely, whether or not the fetus is in distress, yet its value continues to be questioned. With a nonreassuring fetal heart rate pattern, the clinician often needs additional information about fetal oxygen status. Health of the fetus can be assessed intermittently using a Pinard stethoscope or hand-held monitor to listen to the heart rate, or continuously using cardiotocography (CTG), sometimes called electronic fetal monitoring (EFM), or through the recording of the electrical activity of the heart using an electrocardiogram (ECG). Blood samples may be taken from the fetus' head or bottom in a process known as fetal scalp bloodtesting.

Fetal pulse oximetry measures how much oxygen the fetus' blood is carrying. It uses a probe that sits on the baby's head whilst in the uterus and vagina during labor. However, physician access to the fetus is limited and sub-optimal placement of the probe may lead to improper readings.

### SUMMARY

Accordingly, it is a principal object to overcome at least some of the disadvantages of the prior art. In one example, a sensor module is provided for attachment to the mammalian fetus, such as to the forehead, in an improved manner, e.g. a non-invasive manner. The sensor module may comprise multiple light sources of different wavelengths and at least one optical sensor. The light sources may utilize visible and near infrared (IR) wavelengths in the range of 530 to 940 nm. A processing unit in communication with the sensor module is provided to energize and control the multiple light sources and the at least one optical sensor, and process the signals. In one example, pre-processing and post-processing phases are provided prior to determining oxygen saturation levels. The pre-processing phases include a scanning phase to locate the optimal placement of the sensor, and a features extraction phase for improving the accuracy of the determined oxygen saturation levels. The post-processing phase may include a confidence level assessment for each output, to enable the clinician to make informed choices based on the output.

The present examples enable an oximetry device for determining mammalian fetus oxygen levels, the oximetry device comprising: a sensor module comprising multiple light sources of different wavelengths and at least one optical sensor; a processing unit in communication with the sensor module, the processing unit comprising a processor, and a memory, the processor in communication with an output device, the memory comprising electronically readable instructions to cause the processor to: energize the multiple light sources; determine a time varying amplitude of an output signal of the at least one optical sensor; asses a quality of a location of the sensor module responsive to the determined time varying amplitude; provide to the output device an indication of the assessment of quality of the location; extract features from the determined time varying amplitude; and output an indication of the mammalian fetus oxygen level responsive to the extracted features from the determined time varying amplitude.

In some examples, the electronically readable instructions cause the processor to output an indication of pulse rate of the mammalian fetus responsive to the extracted features from the determined time varying amplitude. In some examples, the multiple light sources of different wavelengths comprise wavelengths between 540 nm and 940 nm. In some examples, the sensor module comprises one or more of: a camera; an acoustic sensor; a thermal sensor; a pH sensor; a pressure sensor and an accelerometer.

In some examples, the extracted features comprise a quality of a pulse pattern responsive to a standard deviation of a cyclic height and width of the time varying amplitude. Optionally, the extracted features comprise an influence of blood, the influence of blood responsive to an average cyclic height of the time varying amplitude. Further optionally, the extracted features comprise a signal to noise ratio responsive to the average cyclic height of the time varying amplitude and a cyclic minimum of the time varying amplitude. In one further optional example, the extracted features comprise a noise stability responsive to a standard deviation of the cyclic minimum of the time varying amplitude.

In some examples, the extracted features comprise at least one of: a quality of a pulse pattern responsive to a standard deviation of a cyclic height and width of the time varying amplitude; an influence of blood, the influence of blood responsive to an average cyclic height of the time varying amplitude; a signal to noise ratio responsive to the average cyclic height of the time varying amplitude and a cyclic minimum of the time varying amplitude; and a noise stability responsive to a standard deviation of the cyclic minimum of the time varying amplitude. In some examples, the electronically readable instructions cause the processor to output a confidence level of the output indication of the mammalian fetus oxygen level.

Independently, the present examples provide for a method of determining mammalian fetus oxygen levels, the method comprising: providing a sensor module comprising multiple light sources of different wavelengths and at least one optical sensor for attachment to the mammalian fetus; energizing the multiple light sources; determining a time varying amplitude of an output of the at least one optical sensor; assessing a quality of a location of the sensor module responsive to the determined time varying amplitude; providing to an output device an indication of the assessment of quality of the location; extracting features from the determined time varying amplitude; and outputting an indication of the mammalian fetus oxygen level responsive to the extracted features from the determined time varying amplitude.

In some examples, the method comprises outputting an indication of pulse rate of the mammalian fetus responsive to the extracted features from the determined time varying amplitude. In some examples, the multiple light sources of different wavelengths comprise wavelengths between 530 nm and 940 nm. In some examples, the sensor module comprises one or more of: a camera; an acoustic sensor; a thermal sensor; a pressure sensor; a pH sensor; and an accelerometer.

In some examples, the extracted features comprise a quality of a pulse pattern responsive to a standard deviation of a cyclic height and width of the time varying amplitude. In some further examples, the extracted features comprise an influence of blood, the influence of blood responsive to an average cyclic height of the time varying amplitude. In some yet further examples, the extracted features comprise a signal to noise ratio responsive to the average cyclic height of the time varying amplitude and a cyclic minimum of the time varying amplitude. In some yet further examples, the extracted features comprise a noise stability responsive to a standard deviation of the cyclic minimum of the time varying amplitude.

In some examples, the extracted features comprise at least one of: a quality of a pulse pattern responsive to a standard deviation of a cyclic height and width of the time varying amplitude; an influence of blood, the influence of blood responsive to an average cyclic height of the time varying amplitude; a signal to noise ratio responsive to the average cyclic height of the time varying amplitude and a cyclic minimum of the time varying amplitude; and a noise stability responsive to a standard deviation of the cyclic minimum of the time varying amplitude. In some examples, the method comprises outputting a confidence level of the output indication of the mammalian fetus oxygen level.

Additional features and advantages of the invention will become apparent from the following drawings and description.

### BRIEF DESCRIPTION OF THE DRAWINGS

For a better understanding of certain embodiments and to show how the same may be carried into effect, reference will now be made, purely by way of example, to the accompanying drawings in which like numerals designate corresponding elements or sections throughout.

With specific reference now to the drawings in detail, it is stressed that the particulars shown are by way of example and for purposes of illustrative discussion of the preferred embodiments only, and are presented in the cause of providing what is believed to be the most useful and readily understood description of the principles and conceptual aspects. In this regard, no attempt is made to show structural details in more detail than is necessary for a fundamental understanding, the description taken with the drawings making apparent to those skilled in the art how the several forms may be embodied in practice. In the accompanying drawings:

FIG. 1 illustrates a block diagram of an example oximetry device;

FIGS. 2A-2B illustrate high level flow charts of an example of operation of the oximetry device;

FIGS. 3-4 illustrate operation of the example oximetry device in assessing the quality of location;

FIGS. 5A-5C illustrate graphs of an example reflective oximeter measurement when placing the sensor in a good location, medium location and poor location, respectively;

FIG. 6 illustrates of a test environment.

### DETAILED DESCRIPTION

The present examples enable a novel fetal pulse oximetry and a method for measuring a fetus oxygen level during labor. The device consists of two parts: a sensor module that is inserted through the cervix and attached to the fetus, and a processing unit which is located outside of the cervix. The sensor and the processing unit may be attached by wire or communicate wirelessly using a standard or an embedded communication protocol. The processing unit may receive analog signals from the sensor module, process them, and determine the oxygen level of the fetus. The determined oxygen level is output for display on an output device, such as a designated screen or an existing delivery room screen or monitor. While analog signals may be transmitted from the sensor module to the processing unit, in another example, digital signals, or a combination thereof, may be transmitted.

The sensor module comprises multiple light sources (LEDs) with different wavelengths (the wavelengths may range from 530 nm to 940 nm), and one or more optical sensors, such as a photodiode, suitable for receiving signals from the light sources, i.e. wavelengths produced by the light sources, after interaction with the fetus tissues. The sensor module may contain additional sensors such as a camera, an acoustic sensor (transmitter and receiver), a thermal sensor, a heat sensor, a pressure sensor, a pH sensor, and an accelerometer.

The processing unit is in charge of the operation of the device. It energizes and controls the light sources and the one or more optical sensors, and contains the algorithm that converts the received signals into a number representing the oxygen levels and pulse rate of the fetus. It may also include a time-division and or wavelength-division multiplexing (TDM/WDM) function, signal filters, signal amplifiers, and an analog to digital converter (ADC).

Determining the fetus oxygen level is accomplished in four main phases:

Phase I. Assessing quality of location of the sensor module—In this phase, the processing unit is set on scanning mode and assesses the quality of the current location. The quality is assessed responsive to several parameters, as will be described further below. The practitioner scans a small area of the fetus's skin (around the designated location) with the sensor module, and looks at the output presented on the display device. The practitioner may set the sensor module to the fetus at the location with the highest score. The algorithm may save the maximal score and its location, and may guide the practitioner to that location at the end of the scan (e.g., by showing arrows on the screen or by providing physical signs such as vibrations).

Phase II Extracting features on the chosen location—In this phase, the processing unit is in a pre-processing mode and collects information on the chosen location's behavior, as will be described further below. This is sort of an “on the fly” calibration procedure that helps to improve the accuracy of the oximetry device.

Phase III Determining, on an ongoing basis, the oxygen level of the fetus

Phase IV Assessing the confidence level of the output (also known as post-processing).

Phase I and II may occur only once per patient use, while phases III and IV occur repeatedly.

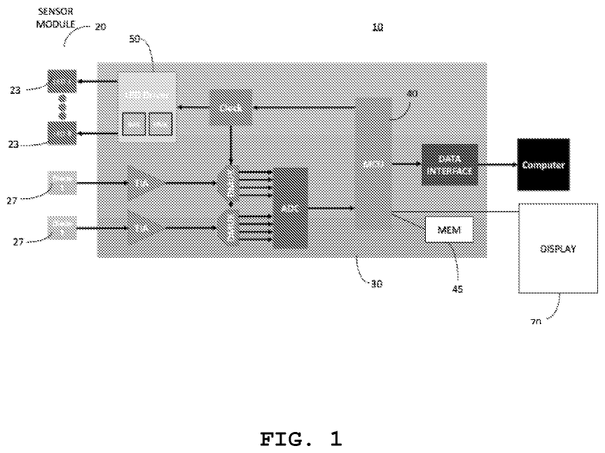

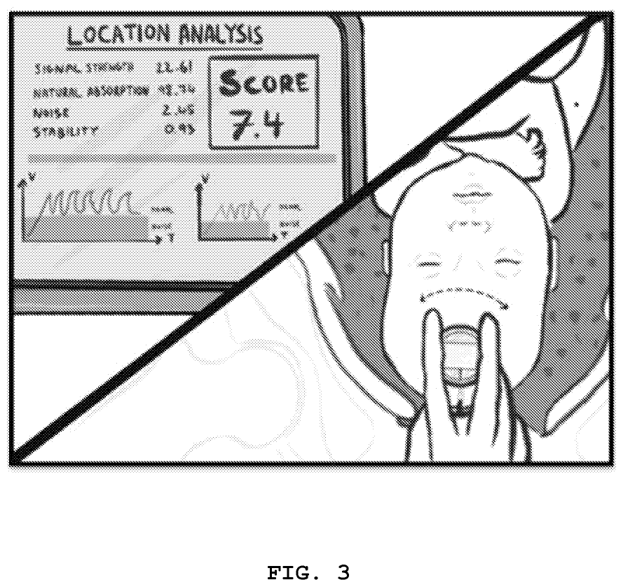

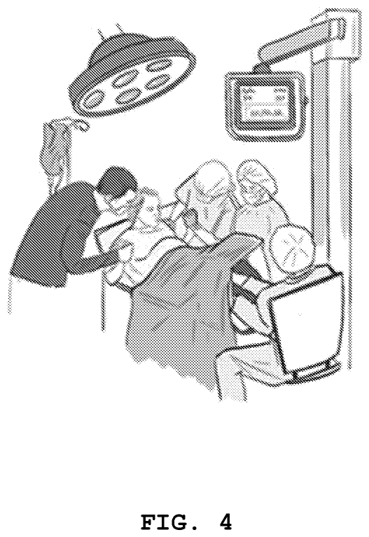

FIG. 1 illustrates a high level block diagram of an example oximetry device 10. Oximetry device 10 comprising a sensor module 20 and a processing unit 30. Sensor module 20 is small and arranged to be inserted through the cervix and attached to the fetus, in one example to the fetus forehead (see FIGS. 3-4). Sensor module 20 may be considered as an internal sensor, is in one example disposable. Sensor module 20 comprises multiple light sources 23 of different wavelengths and at least one optical sensor 27. In one example, light sources 23 comprise light sources outputting light between 530 nm and 940 nm. In one example a first light source 23 is an LED light source outputting light of a nominal 730 nm and a second light source 23 is an LED light source outputting light of a nominal 890 nm.

Processing unit 30 is in communication with sensor module 20 and comprises a processor 40 and a memory 45 in communication with the processor 40. Memory 45 comprises electronically readable instructions to cause the processor perform the actions described herein. Processing unit 30 may comprises an LED driver 50 to control and operate each of multiple light sources 23 and may comprise amplifiers, such as transimpedance amplifiers, to amplify an output of each of the optical sensors 27. A signal generator may be provided, to frequency modulate the light produced by the multiple light sources 23. Processing unit 30 may comprises a multiplexer (time-division and or wavelength-division) and analog to digital converter, with associated clock, to convert the amplified outputs of each of the optical sensors 27 to the digital domain for processing may processor 40. Processing unit 30 may comprise a data interface so as to provide its output to a computer and/or to a display unit 70. Frequency modulation assists in separating the contribution from each of the light sources.

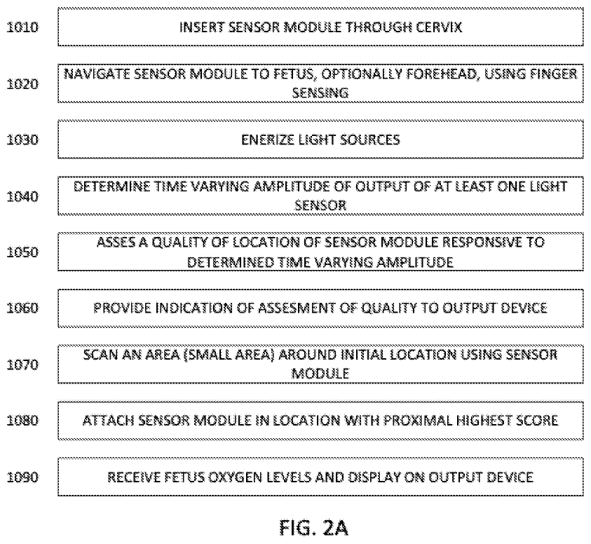

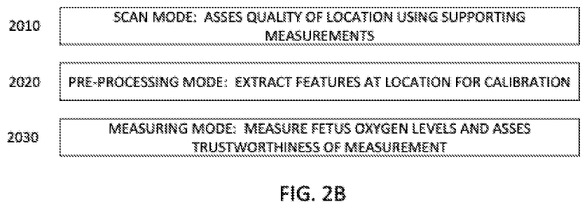

FIG. 2A illustrates a high level flow chart of the operation of oximetry device 10. In stage 1010 sensor module 20 is inserted through the cervix, and in stage 1020, sensor module 20 is navigated to the fetus using finger sensing by the practitioner. In one example sensor module 20 is navigated to the fetus forehead.

In stage 1030, multiple light sources 23 are energized, and in stage 1040, a time varying amplitude of an output of the at least one optical sensor 27 is determined, for example by processor 40. In stage 1050, a quality of the location of the sensor module 30 is assessed responsive to the determined time varying amplitude. In stage 1060 an indication of the assessment of quality of the location is output to the output device, e.g. output device 70. In stage 1070, a small area around the initial location may thus be scanned to find an improved location.

As can be seen in FIGS. 3-4, a small area around the initial location is scanned using sensor module 20 while reviewing the output displayed on display device 70. The location analysis provides a score, as well information such as signal strength, natural absorption, noise, and stability of the signal. Graphs, as described below, may be further shown to assist the practitioner in finding an improved location. In one example the score is a result of multiple parameters, such as signal strength, natural absorption, noise, and stability of the signal.

An advantage of the present embodiments is thus in assisting the practitioner in finding an improved, or optimal, placement location by scanning an area of the fetus's skin. An algorithm that assesses the quality of the current location is provided, as described herein. The algorithm may take into consideration the following parameters:

Photoplethysmography (PPG)—The pulse is important for measuring oxygen saturation because it enables elimination, at least partially, of the effect of absorption caused by the skin and other tissue (which may change between people and affect the result). This happens since the absorption of the skin and otter tissues barely changes in short episodes, while the absorption of the blood is highly affected by the pulse. Given a reflectance graph of one of the LEDs (i.e. the signals captured by the optical sensor 27 from the reflectance of light from one light source 23 on the fetus's skin), processor 40 determines the cyclic pattern of the signals (which represents fetus's pulse). The quality of the pulse's pattern as represented by the cyclic pattern is determined by the standard deviation of the cyclic height and width. Smaller standard deviation outputs reflect more steadiness in the measurement which translates into a higher score.

Since we are interested in measuring the oxygen levels in the fetus's blood, sensor module 20 is preferably placed in a location where the blood's influence is high to provide an accurate PPG. The blood's influence is measured by the average cyclic height.

Signal to noise ratio (SNR)—As indicated above, the blood's influence is our desired signal, while the remaining absorption (due to skin and other tissue) is noise for our measurement. Thus, the SNR is defined here as the cyclic height divided by the cyclic minimum. Higher SNR means less noise interference and therefore higher a score.

Noise stability—One of the assumptions of pulse oximeters is that the absorption levels of the non-blood components remain constant. Venal blood is also considered static and contributes a static component to the PPG. Noise stability is defined herein to be the standard deviation of the cyclic minimum. Higher noise stability means a lower standard deviation of the cyclic minimum and therefore a higher score.

These parameters may be extracted driving several light sources 23, and combined the resulting output of optical sensors 27 together to a single value. Since a good heuristic for the optimal location is to be on an artery, this phase may involve two parts: finding nearby arteries (which may be done using a different technology or sensor, such as a camera) and assessing the quality of each of them.

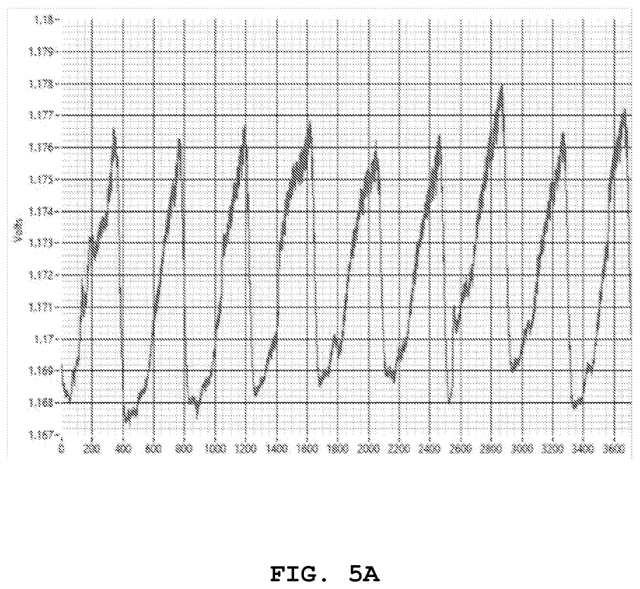

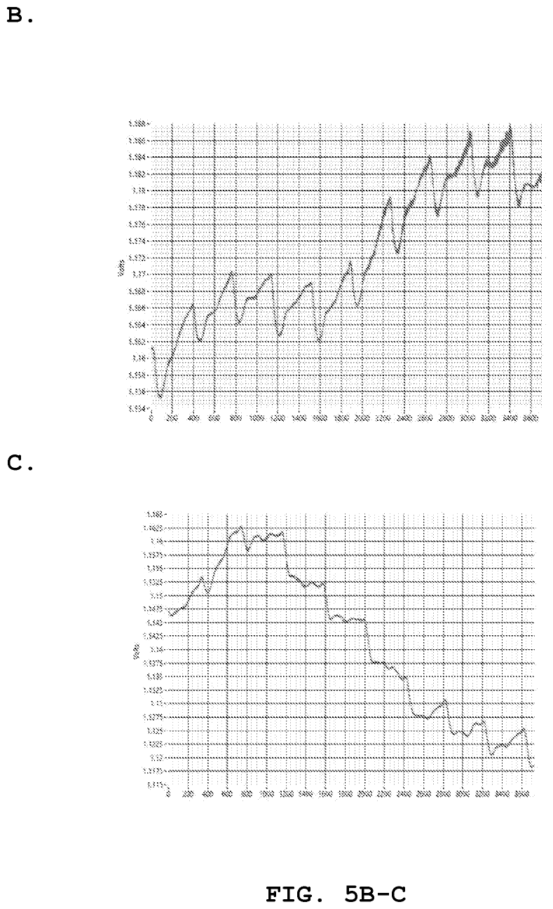

FIG. 5A illustrates a graph of the number of samples vs. amplitude of reflective oximetry device 10 when sensor module 20 is placed in a good location, i.e. a location with stable measurements and a clear pulse reading. FIG. 5B illustrates a graph of the number of samples vs. amplitude of reflective oximetry device 10 when sensor module 20 is placed in a medium location, i.e. a location with unstable measurements and a semi-clear pulse reading. FIG. 5C illustrates a graph of the number of samples vs. amplitude of reflective oximetry device 10 when sensor module 20 is placed in a poor location, i.e. a location with unstable measurements and an unclear pulse reading.

In stage 1080, the sensor module 20 is attached to the fetus at the determined location, i.e. the location with the highest score, or near the highest score, or as best as can be practically achieved. There is no requirement that attachment be achieved at the precise highest score. In stage 1090, features are extracted from the determined time varying amplitude over time, as described further below, and an indication of the mammalian fetus oxygen level responsive to said extracted features from the determined time varying amplitude is output on output device 70.

FIG. 2B illustrates a high level flow chart of receiving fetus oxygen level indications after placements, and extracting features. In stage 2010, the quality of the location is assessed using supporting measurements, as described above in relation to Phase I, as part of a scan mode. This phase may involve similar measurements as performed in phase I (i.e. pulse's pattern, blood's influence, SNR, and noise stability). These features preferably are extracted responsive to each of the individual light sources 23, but may be extracted from a subset of them, and preferably be measured for a longer period of time. Additional features may be extracted from the same measurements, as well as from new measurements which may involve additional components (such as a camera, an acoustic sensor, a thermal sensor, a heat sensor, a pressure sensor, a pH sensor an accelerometer, without limitation).

In stage 2020, as part of a pre-processing mode, features are extracted at the chosen location for calibration. In one example, a window of the PPG graph is analyzed for each wavelength and features are extracted such as: pulse frequency (values, mean, variance), min and max peaks (values, mean, variance). Additional features may be determined in accordance with an Artificial Intelligence algorithm.

In stage 2030 calculation of the fetus's oxygen levels is performed using an algorithm that takes into consideration the extracted features from phase II, including an assessment of the trustworthiness of the measurement. The algorithm extracts the hemoglobin parameters in blood, based on the ratio between signals' parameters in each of the wavelengths, since the absorption spectrum of blood is wavelength-dependent. This procedure is performed after a predetermined number of measurements to assess the quality of the given reading. In addition to providing the most accurate measurement that we can, it is also important for the practitioner to be apprised of our confidence level of a given output, which affects its trustworthiness and the practitioner's reaction upon that. The confidence level is determined by combining various factors which may involve parameters similar to phases I and II (such as pulse's pattern, blood's influence, SNR, and noise stability), and may involve additional factors. The calculation of the confidence level of a given output may be affected by the data of previous outputs (for instance, it is less likely that the oxygen level will have a high change in small periods), so as to avoid high volatility in the result.

The pulse oximetry is specially designed for measuring fetus saturation during labor in several ways 1. The wavelengths utilized, i.e. multiple wavelengths in the range of 730 nm-890 nm, are more suitable in low saturation scenarios (the fetus's saturation level is expected to be significantly lower than for adults). 2. The physics model utilized is tailored for the light's scattering and absorption pattern in such a low saturation scenario, which better captures the presence of other molecules such as Carboxyhemoglobin (COHB) and Methemoglobin (MetHB), PO₂, PCO₂, to improve accuracy. The predetermined distance between the light sensor and the multiple light sources may be utilized. Further, multiple light sources of a particular wavelength may be placed on opposing sides of an optical sensor may be provided to improve the response over areas where the optical characteristics may not be uniform. 3. A unique algorithm and supportive measurements allow us to accurately measure oxygen levels without the need for calibration (which calibration is not possible in this scenario).

The methods described herein allow the practitioner to secure the sensor module in an optimal, or near optimal, location, avoiding errors that occur from placing the sensor module in bad areas (such as with low perfusion for instance). The sensor module is small to enable insertion through the cervix

The present oximetry device measures oxygen saturation levels accurately, even when oxygen levels are low. Thus, the oximetry device may also be used on newborns and adult patients as well, with high or low oxygen levels. The device may also be used on animals.

### Training and Validations:

Since currently there is no way of assessing the oxygen levels of a fetus, it is important to invest in alternative tests and validations. In addition to animal tests, which are also very limited in the oxygens levels one can reach, we have developed a controlled environment that is very close to the real scenario.

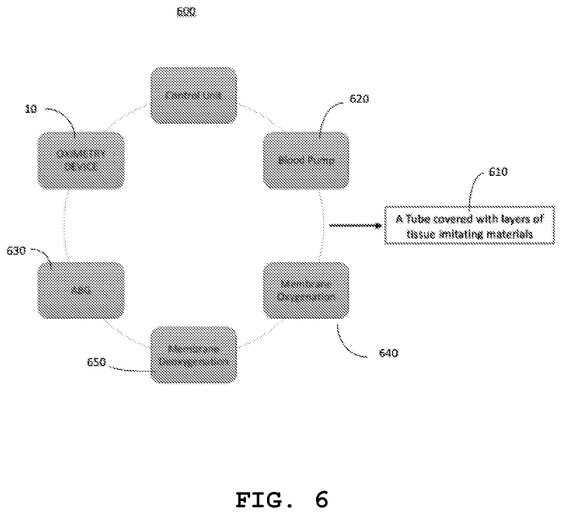

Referring to FIG. 6, the test environment 600 may include the following. A tube 610 in which the blood will flow, covered with layers of material that simulate the fetus's skin and tissues (focusing on the absorption of the used wavelengths). A pump 620 that simulates the heart pulses and pumps the blood through the tube. An Arterial Blood Gas (ABG) analyzer 630 to measure the current oxygen levels of the blood (we may use one which is more suitable for low oxygen levels). A membrane 640 that oxygenates the blood. A membrane 650 that deoxygenates the blood. A control unit is provided that controls the oxygen level in the system by changing parameters in the oxygenator, membrane 640, and deoxygenated components, i.e. membrane 650.

Hemoglobin of an adult human is mostly composed of HbA type (which composed of 2 alpha-globin and 2 beta-globin), while the hemoglobin of a fetus human is mostly composed of HbF type (which composed of 2 alpha-globin and 2 gamma-globin). Different hemoglobin may have different absorption graphs. The test environment 600 is robust for any type of fluid. We may use a synthesized or real blood of a human or an animal. In addition, we may use a fetus's blood (extracted from the umbilical cord).

### Machine Learning and Further Improvements:

When the newborn comes out and starts to breathe, oxygen levels rise rapidly. Our algorithm may involve a learning phase to increase accuracy by getting feedback right after the labor from the fetus's oxygen levels measurement. We may combine supervised and unsupervised machine learning algorithms during our development phase, using data collected from the test environment, animal tests, and clinical trials.

Our oximetry device provides important information on the fetus which may assist a practitioner in making labor-related decisions (such as whether to perform a regular delivery or a C-Section). All the measurements may be saved securely on a remote cloud, together with post-labor information on the fetus. After enough data is collected, machine learning may be utilized to train the algorithm to predict the outcome of the labor (i.e. whether this labor should be delivered vaginal or using a C-Section). The machine learning algorithm can be applied either on our output (oxygen levels) or directly on the raw measurement of the oximetry device. When utilized on raw measurements, the algorithm may capture additional features that may affect whether a C-Section is needed.

The disclosed method for improving oxygen level and pulse measurements can be further generalized to additional measurements such as glucose levels and detecting meconium in amniotic fluid. In addition, to provide a better picture for the practitioner, we can add additional measurements such as internal heat, labor contractions, glucose levels, and pH without limitation.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove. Rather the scope of the present invention is defined by the appended claims and includes both combinations and subcombinations of the various features described hereinabove as well as variations and modifications thereof which would occur to persons skilled in the art upon reading the foregoing description and which are not in the prior art.

1. An oximetry device for determining mammalian fetus oxygen levels, the oximetry device comprising:
a sensor module comprising multiple light sources of different wavelengths and at least one optical sensor;
a processing unit in communication with the sensor module, the processing unit comprising a processor, and a memory, the processor in communication with an output device, the memory comprising electronically readable instructions to cause the processor to:
energize said multiple light sources;
receive an output signal of said at least one optical sensor;
asses a quality of a location of the sensor module responsive to the received output signal of said at least one optical sensor;
provide to the output device an indication of said assessment of quality of the location;
extract features from the determined time varying amplitude; and
output an indication of the mammalian fetus oxygen level responsive to said extracted features from the determined time varying amplitude.

2. The oximetry device according to claim 1, wherein the electronically readable instructions cause the processor to output an indication of pulse rate of the mammalian fetus responsive to said extracted features from the determined time varying amplitude.

3. The oximetry device according to claim 1, wherein said multiple light sources of different wavelengths comprise wavelengths between 530 nm and 940 nm.

4. The oximetry device according to claim 1, wherein the sensor module comprises one or more of: a camera; an acoustic sensor; a thermal sensor; a pH sensor; a pressure sensor and an accelerometer.

5. The oximetry device according to claim 1, wherein the extracted features comprise a quality of a pulse pattern responsive to a standard deviation of a cyclic height and width of a time varying amplitude of the output signal of said at least one optical sensor.

6. The oximetry device according to claim 5, wherein the extracted features comprise an influence of blood, the influence of blood responsive to an average cyclic height of the time varying amplitude.

7. The oximetry device according to claim 6, wherein the extracted features comprise a signal to noise ratio responsive to the average cyclic height of the time varying amplitude and a cyclic minimum of the time varying amplitude.

8. The oximetry device according to claim 6, wherein the extracted features comprise a noise stability responsive to a standard deviation of the cyclic minimum of the time varying amplitude.

9. The oximetry device according to claim 1, wherein the extracted features comprise at least one of:
a. a quality of a pulse pattern responsive to a standard deviation of a cyclic height and width of a time varying amplitude of the output signal of said at least one optical sensor;
b. an influence of blood, the influence of blood responsive to an average cyclic height of the time varying amplitude;
c. a signal to noise ratio responsive to the average cyclic height of the time varying amplitude and a cyclic minimum of the time varying amplitude; and
d. a noise stability responsive to a standard deviation of the cyclic minimum of the time varying amplitude.

10. The oximetry device according to claim 1, wherein the electronically readable instructions cause the processor to output a confidence level of the output indication of the mammalian fetus oxygen level.

11. A method of determining mammalian fetus oxygen levels, the method comprising:
providing a sensor module comprising multiple light sources of different wavelengths and at least one optical sensor for attachment to the mammalian fetus;
energizing said multiple light sources;
assessing a quality of a location of the sensor module responsive to an output of the at least one optical sensor;
providing to an output device an indication of said assessment of quality of the location;
extracting features from the output of the at least one optical sensor; and
outputting an indication of the mammalian fetus oxygen level responsive to said extracted features from the output of the at least one optical sensor.

12. The method according to claim 11, comprising outputting an indication of pulse rate of the mammalian fetus responsive to said extracted features from the output of the at least one optical sensor.

13. The method according to claim 11, wherein said multiple light sources of different wavelengths comprise wavelengths between 530 nm and 940 nm.

14. The method according to claim 11, wherein the sensor module comprises one or more of: a camera; an acoustic sensor; a thermal sensor; a pressure sensor, and an accelerometer.

15. The method according to claim 11, wherein the extracted features comprise a quality of a pulse pattern responsive to a standard deviation of a cyclic height and width of a time varying amplitude of the output of the at least one optical sensor.

16. The method according to claim 15, wherein the extracted features comprise an influence of blood, the influence of blood responsive to an average cyclic height of the time varying amplitude.

17. The method according to claim 16, wherein the extracted features comprise a signal to noise ratio responsive to the average cyclic height of the time varying amplitude and a cyclic minimum of the time varying amplitude.

18. The method according to claim 16, wherein the extracted features comprise a noise stability responsive to a standard deviation of the cyclic minimum of the time varying amplitude.

19. The method according to claim 11, wherein the extracted features comprise at least one of:
a. a quality of a pulse pattern responsive to a standard deviation of a cyclic height and width of a time varying amplitude of the output of the at least one optical sensor;
b. an influence of blood, the influence of blood responsive to an average cyclic height of the time varying amplitude;
c. a signal to noise ratio responsive to the average cyclic height of the time varying amplitude and a cyclic minimum of the time varying amplitude; and
d. a noise stability responsive to a standard deviation of the cyclic minimum of the time varying amplitude.

20. The method according to claim 11, comprising outputting a confidence level of the output indication of the mammalian fetus oxygen level.
